(19) United States
(12) Patent Application Publication
(10) Pub. No.: US 2009/0061450 A1
(43) Pub. Date: Mar. 5, 2009

(54) SYSTEM AND METHOD FOR DIAGNOSIS OF INFECTIOUS DISEASES

(75) Inventor: William Samuel Hunter, Jan Juc (AU)

Correspondence Address: SEED INTELLECTUAL PROPERTY LAW GROUP PLLC, 701 FIFTH AVE, SUITE 5400, SEATTLE, WA, 98104, US

(73) Assignee: MICRONICS, INC., Redmond, WA (US)

(21) Appl. No.: 12/203,715

(22) Filed: Sep. 3, 2008

Related U.S. Application Data
(63) Continuation of application No. PCT/US2007/006521, filed on Mar. 14, 2007.

(30) Foreign Application Priority Data
Mar. 14, 2006 (AU) 2006901314

(51) Int. Cl. C12Q 1/68; C12M 1/34

(52) U.S. Cl. 435/6; 435/287.2

(57) ABSTRACT

A biosafe apparatus is disclosed for assay and diagnosis of respiratory pathogens comprising a nasal sampling device, a single entry, disposable microfluidic cartridge for target nucleic acid amplification, and an instrument with on-board assay control platform and target detection means.

### CROSS-REFERENCE TO RELATED APPLICATION

This application is a continuation of International PCT Patent Application No. PCT/US2007/006521, filed Mar. 14, 2007 (now pending); which claims the benefit of Australia Provisional Patent Application No. 2006901314, filed Mar. 14, 2006. These applications are incorporated herein by reference in their entireties.

### BACKGROUND OF THE INVENTION

1. Field of the Invention

The present invention relates to the general fields of molecular biology and medical science, and more particularly to a system for point-of-care detection of a target nucleic acid.

2. Description of the Related Art

A range of tests exist for the detection of nucleic acid sequences, for example tests for diagnosis of infectious diseases, tests for detection of genes and genetic markers implicated in hereditary diseases, and hereditary testing, among others. Depending on the particular test or method which is applied, there can be wide variation in terms of the cost per test, the accuracy of the test, and the speed at which the test results may be obtained. Present commonly applied tests generally fall in one of two different classes.

In a first class of tests, for many infectious diseases there are rapid tests available which may be procured at low cost. These tests are typically of the type described as lateral flow immunoassays in a dip-stick format. Similar such tests are also widely marketed for home pregnancy testing. Lateral flow immunoassays typically use an antibody immobilized onto a membrane to capture an antigen in the analyte. As part of the immunoassay protocol, a subsequent step then binds an antibody and reporter to the captured antigen in a ‘sandwich’. The presence of the captured antigen in the analyte can then be visually observed, usually as a visible stripe in the test window if the test result is positive. Thus the test result is qualitative in that the presence of a particular infectious disease is provided on either a “Test Positive” or “Test Negative” basis as indicated by the presence or absence of the visible stripe.

A problem with rapid lateral flow immunoassays is that a significant amount of the target antigen must be present in the analyte in order for the antibody-antigen-antibody-label ‘sandwich’ to develop into a visible line. Thus, these types of tests suffer from a lack of sensitivity, and are known to deliver a substantial number of false negative results, particularly when a patient is in the early stages of an infection, and when the amount of a particular antigen or virus in the patient may be low. Moreover, it is in these early stages of detection that it is most important that diagnosis is correctly performed in order to administer an appropriate therapeutic to the patient, or to quarantine the patient to prevent the further spread of the infectious disease to the remainder of the community.

In the second class of tests are the many tests which are now available for clinical laboratories which are based on the detection of nucleic acid molecules. These tests commonly use, for example, nucleic acid based probes and nucleic acid amplification techniques such as the Polymerase Chain Reaction (PCR). For many infectious disease tests, PCR, RT-PCR (Reverse Transcriptase Polymerase Chain Reaction) and rtPCR (real time Polymerase Chain Reaction) based methods have become the “gold standard”, displacing more traditional test formats such as cell culturing. The reason why these tests have become the “gold standard” in many cases is that they allow very low copies of the target nucleic acid sequence of, for example, an infectious agent such as a virus present in a patient sample, to be amplified to a level at which the amplicons may be detected. Thus a patient is able to be correctly diagnosed as positive, even when the level of infectious agent in the patient is low and the patient is in the early stages of infection. Furthermore, PCR, RT-PCR and rtPCR tests are able to deliver accurate qualitative test data indicating the actual amounts of a particular infectious agent which may be present. Such information may be useful to the clinician in terms of deciding on the therapeutic course to be administered, and analyzing the subsequent efficacy of the course of treatment.

A problem with PCR-based clinical laboratory testing in general is the high cost of such tests. These tests typically require expensive reagent kits, highly expensive equipment, and specially trained personnel with expertise in molecular biology in order to be able to be performed correctly. Adequate controls and safeguards must be put in place to prevent false positive results which can arise in the event of sample cross-contamination. Furthermore, for many infectious diseases extensive laboratory safety, containment, and waste handling measures must be put in place to safeguard personnel from the possibility of infection.

Furthermore, there have been recent concerns about the possibility of a pandemic, for example an influenza pandemic related to the H5N1 avian influenza virus. If such a pandemic were to occur, the existing clinical laboratory infrastructure for performing PCR-based tests would likely be overwhelmed, and there would not be sufficient equipment or skilled personnel available to deal with the required test throughput. Further, with the need for clinical laboratory infrastructure and skilled personnel, such laboratory-based test methods do not easily provide for mobile field testing.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF SUMMARY OF THE INVENTION

According to a first aspect, the present invention provides a system for testing for presence of a target nucleic acid, the system comprising:
a sample carrier for carrying a sample to be tested;
a microfluidic cartridge comprising a dock for accepting the sample carrier in a sealed fluidic connection, the cartridge comprising inner works in fluidic connection with the sample carrier, and further comprising at least one ported external hydraulic interface to enable assay control, wherein the cartridge is configured to support a nucleic acid amplification process in which the sample remains contained within the cartridge; and
a control platform instrument for controlling the assay via the at least one ported external hydraulic interface of the cartridge, for optically detecting a result of the nucleic acid amplification process, and optionally for heating and stirring the amplification chamber.

In a second aspect, the apparatus the present invention comprises:

a) A two-piece sample carrier comprising a swab for collecting a sample to be tested, said swab with capture end and extended neck topped by a threaded cap, and a body with compartment for accepting said swab, and further comprising a threaded upper lip and lower tubular nose with axial orifice, said orifice with inner seal;

b) A disposable microfluidic cartridge with external surfaces and with internal works, the microfluidic cartridge further comprising a bridging manifold with first fluidic channel in fluidic connection with a sample receiving receptacle, a means for sealingly accepting the tubular nose of said sample carrier in said sample receiving receptacle, a means for fluidically joining said first fluidic channel to said sample carrier, valve means for introducing and withdrawing lysis reagent to and from said compartment, a means for extracting a target nucleic acid from a sample lysate, a means for eluting a target nucleic acid, an amplification chamber and stirrer means for amplifying a nucleic acid in a sample eluate, a lightpath through said chamber for detecting an amplification product by optical detection means; and,

c) An control platform instrument with means for sealedly engaging and controlling said internal works of said microfluidic cartridge; and, said means for sealingly engaging and controlling comprising at least one ported external hydraulic interface on said microfluidic cartridge.

d) Wherein said means for sealingly accepting the tubular nose of said sample carrier in said sample receiving receptacle, said means for fluidically joining said first fluidic channel to said sample carrier, and said means for sealedly engaging and controlling said internal works are configured to isolate said nasal swab, internal works of said microfluidic cartridge, external surfaces, and instrument, from forward and reverse contamination.

According to a third aspect the present invention provides a method for testing for presence of a target nucleic acid, the method comprising:
docking a sample carrier carrying a sample to be tested into a dock of a microfluidic cartridge;
applying a fluidics technique to move the sample via a sealed fluidic connection from the sample carrier to at least one chamber of the microfluidic cartridge;
conducting a nucleic acid amplification process in which the sample remains contained within the microfluidic cartridge; and
optically detecting a result of the nucleic acid amplification process.

In another embodiment, the method for assaying a biosample for a target nucleic acid comprising:

a) Collecting a sample with a swab and threadedly sealing said swab in a sample compartment in a sample carrier; said sample carrier further with tubular nose with central orifice, said orifice with inner seal; then,

b) Sealingly pressing said sample carrier into a sample receiving receptacle of a microfluidic cartridge, said sample receiving receptacle with piercing means, thereby piercing said inner seal and fluidically joining said sample compartment with a first fluidic channel of said microfluidic cartridge; thereafter,

c) Engaging said microfluidic cartridge in a control platform instrument; and,

d) Sealedly introducing and withdrawing a lysis reagent to and from said sample compartment via said first fluidic channel, thereby forming a sample lysate; and aspirating said lysate into an isolation chamber on said microfluidic cartridge; and therein,

e) Sealedly extracting a target nucleic acid from said sample lysate nucleic acid onto a solid phase matrix, thereby forming a solid phase retentate; and,

f) Sealedly eluting the target nucleic acid from said solid phase matrix, thereby forming an eluate; and further,

g) Sealedly amplifying said target nucleic acid; before,

h) Sealedly detecting amplification products by optical detection means;

i) And further having controlled said steps of the assay by activating electrical and hydraulic control interfaces of said control instrument platform; before finally,

j) Disposing said microfluidic cartridge.

The result of the nucleic acid amplification process may be either the presence or absence of an amplification product, which in turn indicates whether or not the target nucleic acid was present in the sample.

The target nucleic acid targeted by the amplification process may be a nucleic acid of an infectious agent, so that such embodiments of the present invention provide for infectious disease testing. Alternatively, the nucleic acid targeted by the amplification process may be a nucleic acid of a human or animal subject, so that such embodiments of the present invention provide for genetic testing of the subject.

Docking of the sample carrier to the microfluidic cartridge is preferably substantially irreversible, such that the sample carrier can not be undocked with the same ease with which it can be docked. Such embodiments may assist in ensuring that each sample carrier and microfluidic cartridge is used once only. For example, the docking of the sample carrier to the cartridge may be achieved by a one way snap-fit arrangement, such that the sealed fluidic connection between the sample carrier and the cartridge can only be established by effecting the one way snap-fit. In such embodiments the sample carrier may comprise one or more resiliently flexible barbs constituting a male part of the dock, to be captured by a matching recess of the microfluidic cartridge constituting a female part of the dock.

The sample is preferably contained within the sample carrier in a bio-safe manner until docking of the sample carrier to the microfluidic cartridge is effected. The sealed fluidic connection between the sample carrier and the microfluidic cartridge may be provided by a needle of the microfluidic cartridge piercing the sample carrier. Preferably, the needle or sharp is recessed or is retracted prior to docking and is mounted such that it advances to pierce the sample carrier only upon docking being effected. The dock preferably encompasses the needle to ensure sealing of the fluidic connection provided by the needle.

Transfer of the sample from the sample carrier to the microfluidic cartridge may be effected by aspiration applied by way of the externally ported hydraulic control interface of the microfluidic cartridge. Prior to transfer, the sample may be lysed by causing flow of a fluid lysis buffer into the sample carrier to lyse the sample. For example guanidinium isothiocyanate may be used as a lysis buffer to enable RNA to be extracted from the sample.

The at least one chamber of the microfluidic cartridge preferably comprises a nucleic acid isolation chamber. The nucleic acid isolation chamber preferably comprises a surface to which the target nucleic acid will attach. For example, the solid phase extraction chamber may be pre-loaded with solid phase particles, such as silica beads, having a surface treatment to which the target nucleic acid binds. Where the nucleic acid is attached in this manner, some or all of the remainder of the lysed sample and the lysis buffer itself may be washed away by a wash buffer. Thus, in such embodiments, the microfluidic cartridge preferably further comprises a waste chamber in fluidic connection with the nucleic acid isolation chamber, for storing such waste material washed away from the nucleic acid. Further, in such embodiments, after washing the nucleic acid is preferably eluted from the solid phase material by the introduction of a suitable elution buffer, for example TRIS.

The microfluidic cartridge preferably further comprises an amplification test chamber. In embodiments comprising a nucleic acid isolation chamber, the amplification test chamber is preferably in fluidic connection with the nucleic acid isolation chamber. The amplification chamber is preferably pre-equipped with a stirrer to mix the sample template with oligonucleotide primers or the like which may be introduced via port(s) of the microfluidic cartridge. Preferably, the stirrer is substantially transparent so as not to obstruct optical detection of test results. The stirrer may comprise at least one magnet to provide for magnetic control of the stirrer. In such embodiments the control platform preferably comprises a magnetic stirrer controller. The target sequence, if present, is then amplified to a level whereby the presence of the target sequence may be rapidly detected using one of a range of detection methods, such as turbidimetric detection, or fluorescence detection.

The microfluidic cartridge preferably further comprises a positive control amplification chamber, and preferably further comprises a negative control amplification chamber. Each such chamber is preferably provided with a respective stirrer.

The microfluidic cartridge is preferably formed of transparent material at least in the vicinity of the amplification test chamber, to enable optical detection of the result of the nucleic acid amplification process. The control platform may optically detect the result of the nucleic acid amplification process by monitoring an intensity of a light signal transmitted through the amplification test chamber, for example where turbidity in the amplification test chamber arises as a result of amplification of the target nucleic acid (a positive test). Additionally or alternatively the control platform may optically detect the result of the nucleic acid amplification process by monitoring for optical emissions at a first wavelength which arise as a result of excitation of a fluorophores in the amplification test chamber by light of a second wavelength, such fluorophores arising in the event of a positive test.

Thus, embodiments of the present invention provide for a microfluidic cartridge which enables nucleic acid amplification techniques to be performed in a sealed environment to provide for containment of potentially hazardous biological samples and amplicons. Embodiments of the invention exploit fluidics techniques by applying fluid flows and aspiration conditions to the port(s) of the microfluidic cartridge.

The system preferably further comprises temperature control means to provide for suitable temperature conditions for the particular nucleic acid amplification process applied. In some embodiments, the microfluidic cartridge may comprise a printed circuit for resistive heating when a current is passed through the printed circuit. In such embodiments the control platform preferably comprises electrical contacts for applying a suitable current through the printed circuit of the microfluidic cartridge to produce the necessary temperature conditions within the amplification chamber. Such an arrangement is advantageous in maintaining control complexity within the control platform while providing a simple heating mechanism upon the microfluidic cartridge.

Additionally or alternatively, the microfluidic cartridge may comprise a heating chamber proximal to and fluidly separate from the amplification chamber, with accompanying ports to provide for circulation of heating fluid through the heating chamber. Such embodiments provide for the control platform to generate heating fluid at a suitable temperature and to circulate the heating fluid through the heating chamber of the microfluidic cartridge. Heat from the heating fluid may be conducted to the amplification chamber to thus control a temperature of the amplification chamber. Temperature sensors may be mounted upon the microfluidic cartridge to provide temperature feedback to the control platform to control the temperature of the heating fluid.

The amplification process may be an isothermal amplification process. Use of an isothermal amplification process may be advantageous in simplifying temperature control requirements of the system. A particularly applicable isothermal amplification process may be the LAMP process (Loop-mediated Isothermal Amplification) manufactured by Eiken Chemical Co., of Tokyo, Japan. Additionally or alternatively, the microfluidic cartridge may support an alternate amplification process such as a different isothermal protocol, or a thermal cycling protocol. Such protocols could be polymerase chain reaction (PCR), ligase chain reaction, Q.beta. replicase, strand displacement assay, transcription mediated iso CR cycling probe technology, nucleic acid sequence-based amplification (NASBA) and cascade rolling circle amplification (CRCA),

In preferred embodiments the microfluidic cartridge is a single-use consumable, and the sample carrier is a single-use consumable. Such embodiments enable the control platform to accept a succession of microfluidic cartridges and to control the execution of a nucleic acid amplification process within each microfluidic cartridge, without the control platform itself coming into contact with potentially bio-hazardous material and thus without the need for the control platform to be located within a bio-safe containment facility. After completion of a test, the single-use microfluidic cartridge and sample carrier may be disposed of in a bio-safe manner. Thus, the microfluidic cartridge and sample carrier are preferably made of inexpensive materials and made to be of a small size to minimise the cost and waste associated with such single-use consumables. A small microfluidic cartridge providing an amplification chamber of small volume is further advantageous in minimising a volume of reagent(s) required for the nucleic acid amplification process, such that a given reagent supply of the control platform may provide for an increased number of tests by the control platform.

Embodiments of the present invention may thus provide for detection of one or more of a range of nucleic acid target sequences, for example for a variety of infectious diseases. Embodiments of the invention may provide for a bio-contained determination of the presence of an infectious disease using a single relatively low-cost instrument. The system is preferably portable and/or located at a point of care, such that test results can be obtained more rapidly on site, while nevertheless using a sensitive and accurate amplification test.

Aspiration and fluid flow paths within the microfluidic cartridge are preferably effected by at least one valve of the microfluidic cartridge, the at least one valve being controllable by the control platform.

The sample carrier is preferably adapted to be sealed after the sample is placed in the sample carrier, until becoming docked with the microfluidic cartridge. For example, the sample may be obtained by a sample swab, with the sample swab being sealed within the sample carrier by closing a one way threaded closure of the sample carrier. The sample swab may be attached to the closure to ensure placement of the sample at a desired location within the sample carrier.

The sample may be mucus obtained by a nasal or throat swab. The sample may additionally or alternatively comprise a biological sample derived from an agricultural source, a bacterial source, a viral source, a human source or an animal source. The sample may additionally or alternatively comprise waste water, drinking water, agricultural products, processed foodstuff, air, blood, stool, sputum, buccal material, serum, urine, saliva, teardrop, a biopsy sample, an histological tissue sample, a tissue culture product, an agricultural product, or the like.

### BRIEF DESCRIPTION OF THE DRAWINGS

Embodiments of the invention will now be described with reference to the accompanying drawings, in which:

FIG. 1 is a perspective view of a sample collection device in accordance with an embodiment of the invention.

FIG. 2 is a perspective view of the sample collection device of FIG. 1 fitted to a closed sample tube to form a sample carrier in accordance with an embodiment of the invention.

FIG. 3 is a perspective magnified view of the outlet end of the sample carrier of FIG. 2.

FIG. 4 is a perspective view of a disposable single-use microfluidics microfluidic cartridge in accordance with an embodiment of the present invention, to which the sample carrier of FIG. 2 has been docked.

FIG. 5 is a plan view of a control platform instrument or reader in accordance with an embodiment of the present invention into which the microfluidics microfluidic cartridge of FIG. 4 has been loaded.

FIG. 6 is a plan view of the microfluidics microfluidic cartridge of FIG. 4.

FIG. 7 is a plan view of the microfluidics microfluidic cartridge of FIG. 4 illustrating sample lysis.

FIG. 8 is a plan view of the microfluidics microfluidic cartridge of FIG. 4 illustrating RNA extraction.

FIG. 9 is a plan view of the microfluidics microfluidic cartridge of FIG. 4 illustrating disposal of waste.

FIG. 10 is a plan view of the microfluidics microfluidic cartridge of FIG. 4 illustrating RNA elution.

FIG. 11 is a plan view of the microfluidics microfluidic cartridge of FIG. 4 illustrating the addition of master mix, stirring, amplification, and detection of the target nucleic acid sequence.

FIG. 12 is a magnified cross section of the microfluidics microfluidic cartridge of FIG. 4 when loaded into the control platform of FIG. 5, illustrating turbidimetric detection.

FIG. 13 is a magnified cross section of the microfluidics microfluidic cartridge of FIG. 4 when loaded into a different embodiment of the control platform, illustrating fluorescence detection.

FIG. 14 is a magnified cross section of a microfluidics microfluidic cartridge in accordance with another embodiment of the invention, illustrating an alternative method of heating the amplification and detection chamber.

FIG. 15 is a block diagram of a diagnostic system in accordance with an embodiment of the present invention.

FIG. 16 is a plan view of the instrument of FIG. 5 after the test has been completed.

### DETAILED DESCRIPTION OF THE INVENTION

The term “oligonucleotide” as used herein refers to a polymer composed of a multiplicity of nucleotide residues (deoxyribonucleotides or ribonucleotides, or related structural variants or synthetic analogues thereof) linked via phosphodiester bonds (or related structural variants or synthetic analogues thereof). Thus, while the term “oligonucleotide” can refer to a nucleotide polymer in which the nucleotide residues and linkages between them are naturally occurring, it will be understood that the term also includes within its scope various analogues including, but not restricted to, single-stranded synthetic primers, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 10 to 30 nucleotide residues, but the term can refer to molecules of any length, although the term “polynucleotide” or “nucleic acid” is typically used for large oligonucleotides.

By “primer” is meant an oligonucleotide which, when paired with a nucleotide strand, is capable of initiating the synthesis of a primer extension product in the presence of a suitable polymerase. The primer is preferably single-stranded for maximum efficiency in amplification but can alternatively be double-stranded. A primer must be sufficiently long to prime the synthesis of extension products in the presence of the polymerase. The length of the primer depends on many factors, including application, temperature to be employed, template reaction conditions, other reagents, and source of primers. For example, depending on the complexity of the target sequence, the oligonucleotide primer typically contains 10 to 35 or more nucleotide residues, although it can contain fewer nucleotide residues. Primers can be large polynucleotides, such as from about 200 nucleotide residues to several kilobases or more. Primers can be selected to be “substantially complementary” to the sequence on the template to which it is designed to hybridise and serve as a site for the initiation of synthesis. By “substantially complementary”, it is meant that the primer is sufficiently complementary to hybridise with a target polynucleotide. Preferably, the primer contains no mismatches with the template to which it is designed to hybridise but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridise therewith and thereby form a template for synthesis of the extension product of the primer.

“Isolation” of a nucleic acid is to be understood to mean a nucleic acid which has generally been separated from other components with which it is naturally associated or linked in its native state. Preferably, the isolated polynucleotide is at least 50% free, more preferably at least 75% free, and more preferably at least 90% free from other components with which it is naturally associated. The degree of isolation expressed may relate to purity from interfering substances.

“Isolation” of a biosample refers to “forward isolation”, wherein the biosample container may be handled without exposure to infectious agent, and to “reverse isolation”, wherein the sample is not contaminated during handling. “Biosafe” thus has a second dimension, assurance of the quality of the sample.

Any method of nucleic acid amplification may be suitable for use in embodiments of the present invention. For example, an isothermal amplification technique may be particularly applicable in the amplification of nucleic acids in the present invention. One such isothermal technique is LAMP (loop-mediated isothermal amplification of DNA) and is described in Notomi, T. et al. Nucl Acid Res 2000 28:e63.

Strand Displacement Amplification (SDA) is another method of carrying out isothermal amplification of nucleic acids which involves multiple rounds of strand displacement and synthesis, i.e., nick translation (Walker et al. Nucleic Acids Research, 1992:1691-1696). A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridised to DNA that is present in a sample. Upon hybridisation, the reaction is treated with RNase H, and the products of the probe identified as distinctive products that are released after digestion. The original template is annealed to another cycling probe and the reaction is repeated.

An exemplary nucleic acid amplification technique is the polymerase chain reaction (referred to as PCR) which is described in detail in U.S. Pat. Nos. 4,683,195, 4,683,202 and 4,800,159, Ausubel et al. Current Protocols in Molecular Biology, John Wiley and Sons, Baltimore, Md. (1989), and in Innis et al., (“PCR Protocols”, Academic Press, Inc., San Diego Calif., 1990). Polymerase chain reaction methodologies are well known in the art. Briefly, in PCR, two primer sequences are prepared that are complementary to regions on opposite complementary strands of a target sequence. An excess of deoxynucleoside triphosphates are added to a reaction mixture along with a DNA polymerase, e.g., Taq polymerase. If the target sequence is present in a sample, the primers will bind to the target and the polymerase will cause the primers to be extended along the marker sequence by adding on nucleotides. By raising and lowering the temperature of the reaction mixture, the extended primers will dissociate from the template to form reaction products, excess primers will bind to the template and to the reaction products and the process is repeated. By adding fluorescent intercalating agents, PCR products can be detected in real time.

Another nucleic acid amplification technique is reverse transcription polymerase chain reaction (RT-PCR). First, complementary DNA (cDNA) is made from an RNA template, using a reverse transcriptase enzyme, and then PCR is performed on the resultant cDNA.

Another method for amplification is the ligase chain reaction (“LCR”), disclosed in EPO No. 320 308. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qβ Replicase, may also be used as still another amplification method in the present invention. In this method, a replicative sequence of RNA that has a region complementary to that of a target is added to a sample in the presence of an RNA polymerase. The polymerase will copy the replicative sequence that can then be detected.

Still further amplification methods, described in GB Application No. 2 202 328, and in PCT Application No. PCT/US89/01025, may be used in accordance with the present invention. In the former application, “modified” primers are used in a PCR-like, template- and enzyme-dependent synthesis. The primers may be modified by labelling with a capture moiety (e.g., biotin) and/or a detector moiety (e.g., enzyme). In the latter application, an excess of labelled probes are added to a sample. In the presence of the target sequence, the probe binds and is cleaved catalytically. After cleavage, the target sequence is released intact to be bound by excess probe. Cleavage of the labelled probe signals the presence of the target sequence.

Other nucleic acid amplification procedures include transcription-based amplification systems (TAS), including nucleic acid sequence based amplification (NASBA) and 3SR (Kwoh et al., 1989, Proc. Natl. Acad. Sci. U.S.A., 86: 1173; Gingeras et al., PCT Application WO 88/10315). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerisation, DNA/RNA hybrids are digested with RNase H while double stranded DNA molecules are heat denatured again. In either case the single stranded DNA is made fully double stranded by addition of second target specific primer, followed by polymerisation. The double-stranded DNA molecules are then multiply transcribed by an RNA polymerase such as T7 or SP6. In an isothermal cyclic reaction, the RNAs are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 disclose a nucleic acid amplification process involving cyclically synthesising single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase D, resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al. in PCT Application WO 89/06700 disclose a nucleic acid sequence amplification scheme based on the hybridisation of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, M. A., In: “PCR Protocols: A Guide to Methods and Applications”, Academic Press, N.Y., 1990; Ohara et al., 1989, Proc. Natl. Acad. Sci. U.S.A., 86: 5673-567).

Methods based on ligation of two (or more) oligonucleotides in the presence of nucleic acid having the sequence of the resulting “di-oligonucleotide”, thereby amplifying the di-oligonucleotide, may also be used in the amplification step of the present invention. Wu et al., (1989, Genomics 4: 560).

Solid supports suitable for immobilizing nucleic acids are well known in the art and include, but are not limited to, silica-based membranes, nylon, Teflon, beads including polystyrene/latex beads, latex beads, silica beads or any solid support possessing an activated carboxylate, sulfonate, phosphate or similar activatable group, porous membranes possessing pre-activated surfaces which may be obtained commercially (e.g., Pall Immunodyne Immunoaffinity Membrane, Pall BioSupport Division, East Hills, N.Y., or Immobilon Affinity membranes from Millipore, Bedford, Mass.). Optionally, gas plasma treatments are useful in preparing a binding surface.

The “target nucleic acid” means a nucleotide sequence that may be deoxyribonucleic acid (DNA) or ribonucleic acid (RNA) (including ribosomal ribonucleic acid (rRNA), poly(A)+ mRNA, transfer RNA, (tRNA), small nuclear (snRNA), small interfering (siRNA), telomerase associated RNA, ribozymes etc.) whose presence is of interest and whose presence or absence is to be detected in the test.

Infectious agents may include viruses, bacteria, fungi, yeast, Mycoplasma, and the like.

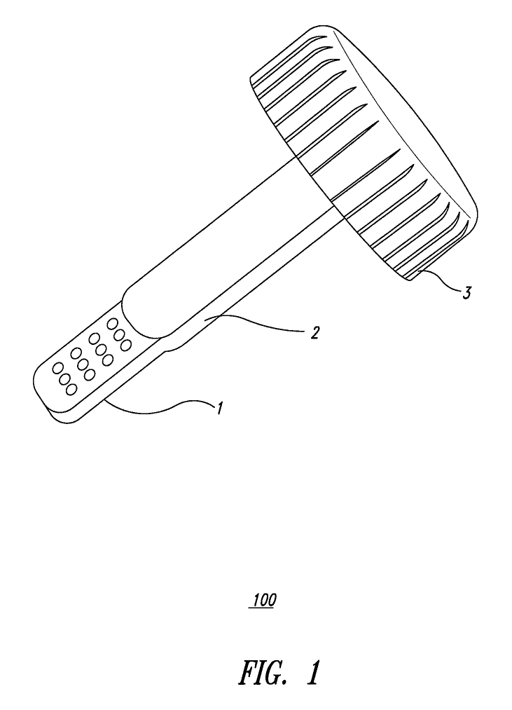

FIG. 1 shows a nasal swab device 100 for human or veterinary application, which may also be used as a throat swab for human nasal swabs, or for animal nasal swabs, or for avian throat swabs. The sample collection is performed by inserting the capture end 1 of the swab into the nostrils of the subject, and briefly rotating the swab in order to collect a mucosal sample. The capture end of the swab 1 is coated with a fibrous material such as Dacron fiber to enhance sample collection efficiency.

The capture end 1 of the swab is connected to a cylindrical neck extension 2. Different variants of the nasal swab device 100 may be manufactured with different lengths of the cylindrical neck extension 2 in order to accommodate different subject types. For example, different length swabs may be required for children compared to adults. Similarly, different length swabs would be required for human, animal, and avian applications.

Cylindrical extension neck 2 is connected to a closure 3. Closure 3 incorporates a ratcheting thread (not shown), similar to those used in child-proof packaging applications, but not re-openable in normal use. Threaded closure 3 also incorporates a gasket element (not shown) on the underside of the cap to provide an air and liquid tight seal when the nasal swab device 100 is fitted to a sample collection tube.

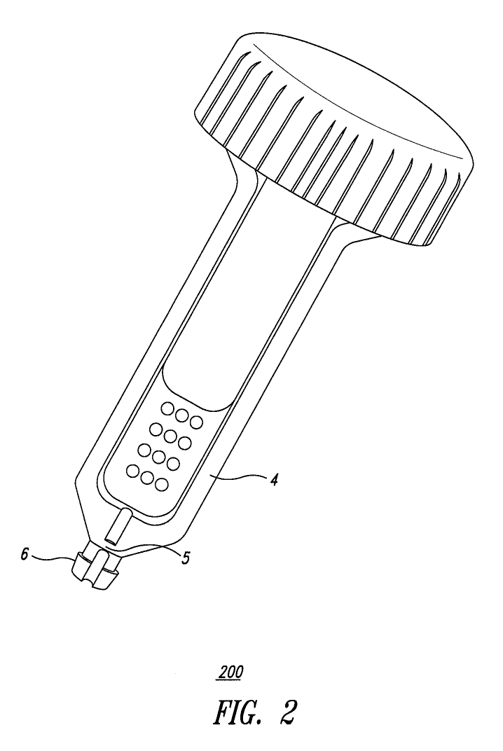

After the nasal or throat swab sample has been collected, the nasal swab is screw-threadingly connected to a sample carrier or sample collection tube 4 as shown in FIG. 2. The screw thread on the sample tube 4 only allows a one-way single use application, such that after the nasal swab device 100 has been fitted to the sample tube 4, it is not possible to unscrew the nasal swab device 100 from the sample collection tube 4. Furthermore, when the nasal swab device 100 is fitted, the gasket element on the underside of the closure 3 seals to the upper circumferential extremity of the sample tube 4 in an air and liquid tight manner.

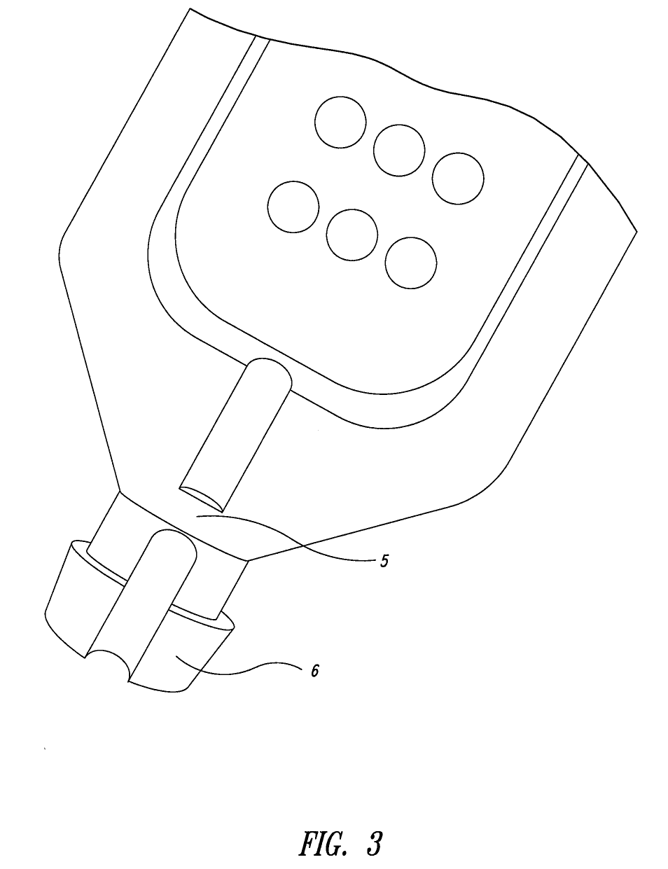

The sample tube 4 is also closed at the outlet end by an integrally molded membrane element or “inner seal” 5 as shown in FIG. 3. Thus when the nasal swab device 100 has been fitted to the sample tube 4, the sample tube assembly 200 is in a bio-safe condition.

The tubular nose 4 of the sample carrier adjacent to the membrane element 5 (internal) also include one or a multiplicity of one-way snap-fit barbs 6 arrayed in a circular manner around the outlet. The purpose of these barbs is to allow the sample tube assembly 200 to be fitted to a microfluidics cartridge in a single-use manner such that bio-safety is maintained.

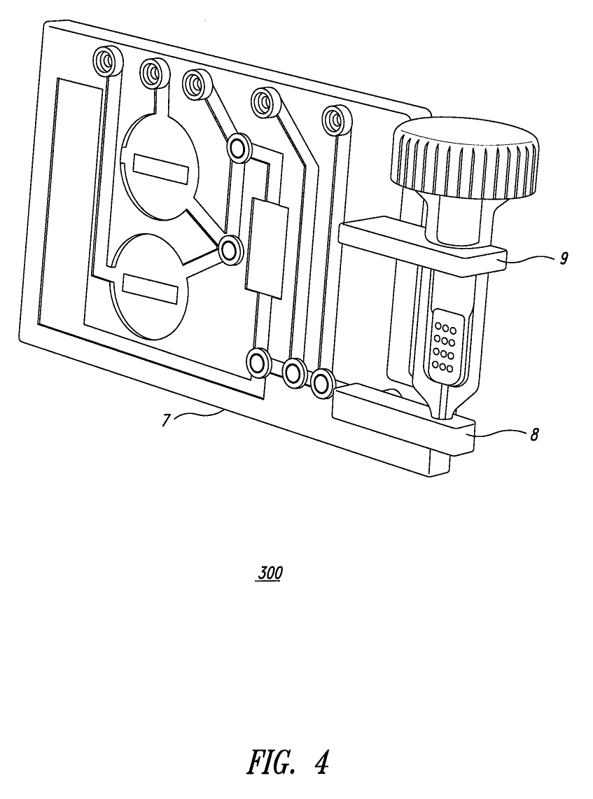

FIG. 4 shows the sample tube assembly fitted to a microfluidic cartridge 7 to create a microfluidics cartridge assembly 300.

Sample tube assembly 200 is inserted through a bridging support element or “docking clamp” 9, which is mounted to the microfluidic cartridge 7. This docking clamp provides structural integrity to the connection of the sample tube assembly 200 with the microfluidic cartridge 7. After the sample tube assembly 200 is inserted through the docking clamp 9, the outlet end of the sample collection tube 4 is inserted into a mating hole within a bridging manifold element 8. When the one way snap-fit barbs 6 near the outlet end of the tube enter the manifold element 8, an undercut female locking ring near the entrance of manifold element sample receiving receptacle 8 (not shown) causes the one way snap-fit barbs 6 to compress and then snap back in such a manner that the sample collection tube is then irreversibly and tightly captured as part of the microfluidics cartridge assembly 300. Such methods of providing a one-way snap-fit using flexible plastic retaining elements are well known to those skilled in the art.

Further insertion of the sample tube assembly 200 into the bridging manifold element 8 causes a small shielded needle or chevron (not shown) within manifold element 8 to puncture the integral plastic membrane element 5 at the outlet end of sample tube 4. The manifold element 8 has an internal fluid passage (not shown) which thereby fluidly interconnects the pierced sample collection tube 4 with the microfluidic cartridge 7 in a leak-tight manner which does not compromise bio-safety. The sample tube assembly 200 is thus coupled to the manifold 8 in a bio-safe and non-releasable manner which enables the sample contents with the sample tube assembly 200 to be analyzed within the microfluidic cartridge 7.

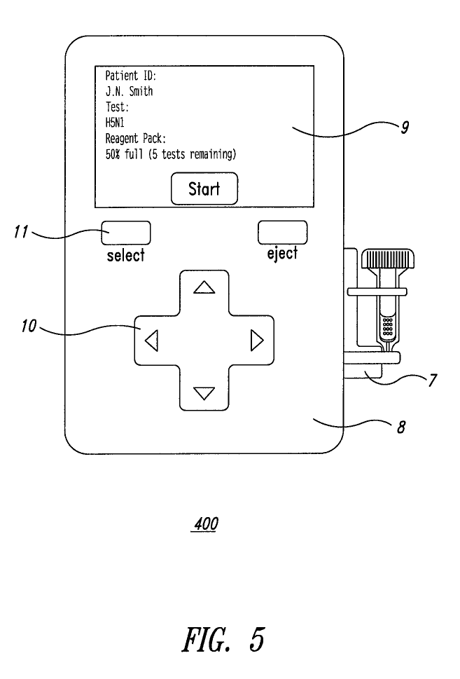

FIG. 5 shows the microfluidics cartridge assembly 300 inserted into an instrument or reader 8 which is capable of performing a number of pre-determined assay steps on the microfluidic cartridge assembly 300. The instrument 8 is controlled by an internal microprocessor, with a user interface displayed on a liquid crystal display (LCD) device 9, and with various parameters on a menu accessible via a four way toggle button 10 and with select button 11. In addition to providing the fluid transport means for the microfluidics cartridge assembly 300, the instrument 8 also contains a reagent pack (not shown) which is capable of dispensing various reagents and buffers to the microfluidics cartridge assembly 300 in accordance with a pre-determined assay protocol stored in the memory of instrument 8, and running under the control of the instrument's microprocessor.

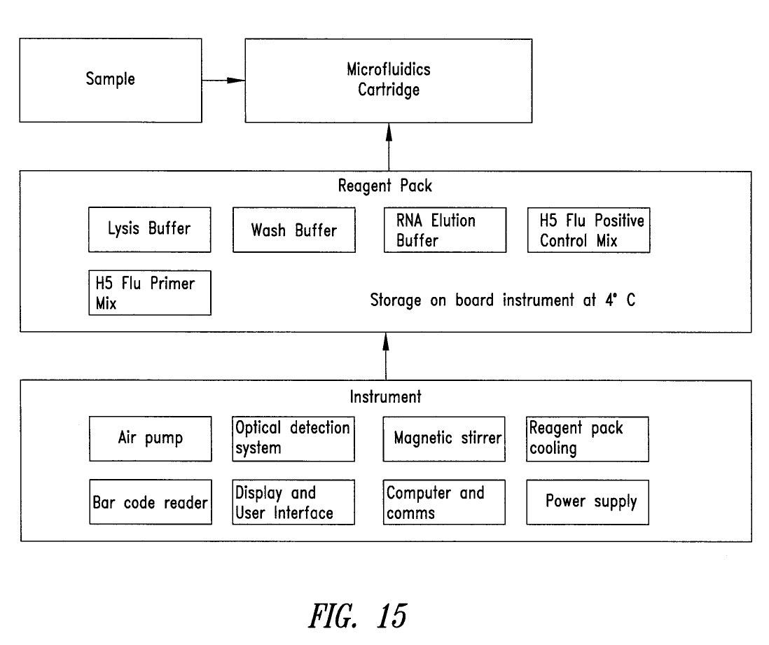

FIG. 15 shows the key elements of the instrument and reagent pack in block diagram format using the example of a reagent pack for a test for H5 avian influenza. The purpose of these various elements shown in this block diagram will become apparent in subsequent description.

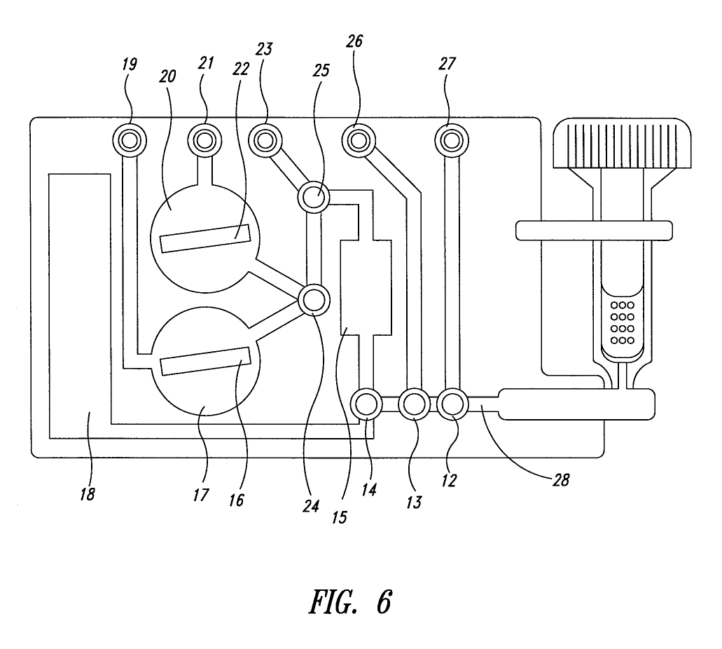

FIG. 6 shows the key elements of the “inner workings” of the microfluidics cartridge assembly 300. Fluid transport around microfluidics cartridge assembly 300 is accommodated by the layout of various microfluidic channels embedded inside the cartridge, such as microfluidic channel 28. The logic for the control of fluid transport around the cartridge is accommodated by the use of various valves embedded in the cartridge, here valves 12, 13, 14, 24, and 25. These valves are shown in FIG. 6 as 3-way valves, however the 3-way valve logic could also be replaced by an increased number of embedded simpler and cheaper 2-way elastomeric valves, which are well known to those skilled in the art of microfluidics design.

Also shown in FIG. 6 are a number of ports 19, 21, 23, 26 and 27 of the externally ported hydraulic control interface of the microfluidic cartridge. These ports each enable a fluid tight connection between microfluidic cartridge 7 when assembled in the control platform instrument 8. The ports and hydraulic control interface enables various reagents to be delivered from the reagent pack stored in instrument 8 to the microfluidics cartridge assembly 300. Some of the ports only enable an air volume to be aspirated or dispensed in order to allow the biohazardous sample material to be transported only within the microfluidics cartridge assembly 300 without ever breaching any of the ports. This ensures that biohazardous infectious material is always contained solely within the cartridge assembly 300.

Further shown in FIG. 6 are inner workings comprising a solid phase extraction chamber 15, a waste containment chamber 18, a test amplification and detection chamber 20, a positive control amplification and detection chamber 17, and micro-magnetic stirrer bar elements 16 and 22 and waste disposal chamber 18. The purpose of these elements will become apparent in subsequent description.

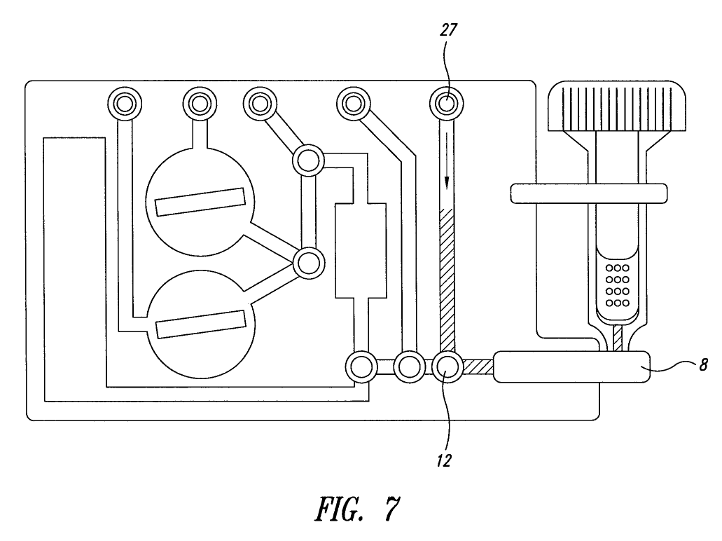

FIG. 7 shows the first step of the pre-programmed assay controlled by instrument 8, which is the introduction of a lysis buffer with for example guanidinium thiocyanate in combination with detergents (shown cross-hatched) from a reagent pack (not shown) through port 27 via valve 12, and via bridging manifold 8 back into sample collection tube 4. This step causes the lysis buffer to mix with the mucosal sample, thereby lysing the cells contained therein, and causing the nucleic acids within the cellular material to be released.

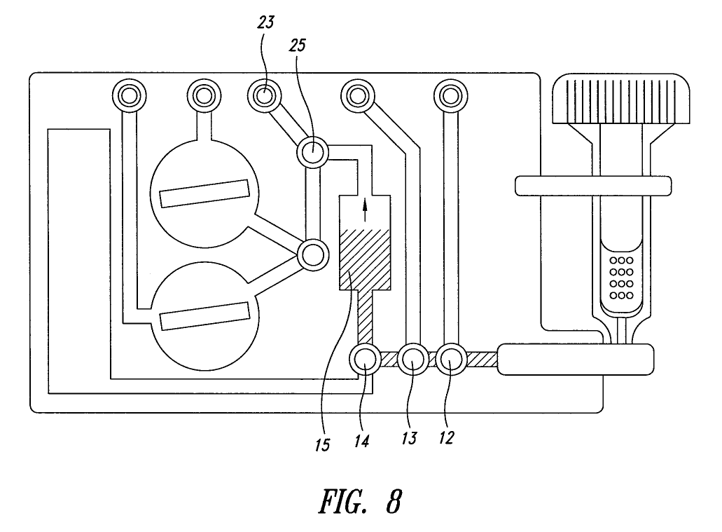

FIG. 8 shows the second step of the pre-programmed assay controlled by instrument 8, which is the aspiration of the lysed sample (shown cross-hatched) from the sample collection tube 4 via bridging manifold 8, and via valves 12,13, and 14 into the RNA isolation chamber 15. Aspiration is applied by way of port 23 and valve 25. The RNA isolation chamber 15 is filled at fabrication with solid phase material such as silica particles which have a surface treatment which will bind only the sample RNA to the surface of the solid phase material. Such solid phase materials are well known to those skilled in the art, and such solid phase materials are available from a range of different manufacturers.

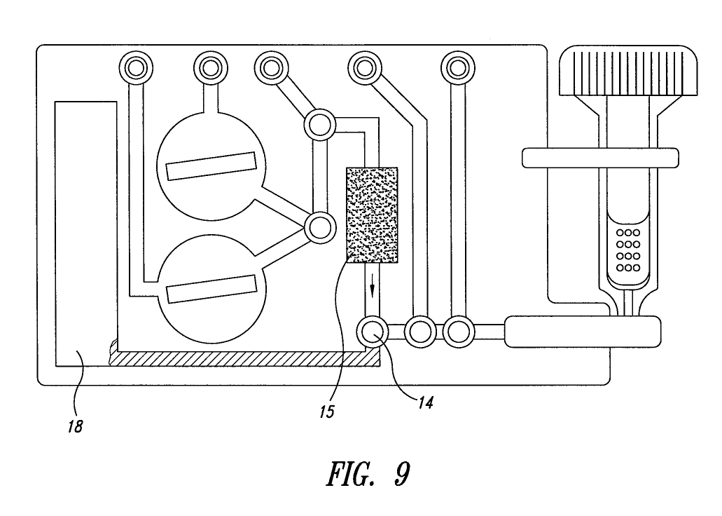

FIG. 9 shows the third step of the pre-programmed assay controlled by instrument 8, which is the elution of waste material from the sample (that is, everything except for the sample RNA) via valve 14 to the waste disposal chamber 18. The eluted waste material is shown cross-hatched, while the remaining captured RNA inside isolation chamber 15 is shown in a dotted pattern.

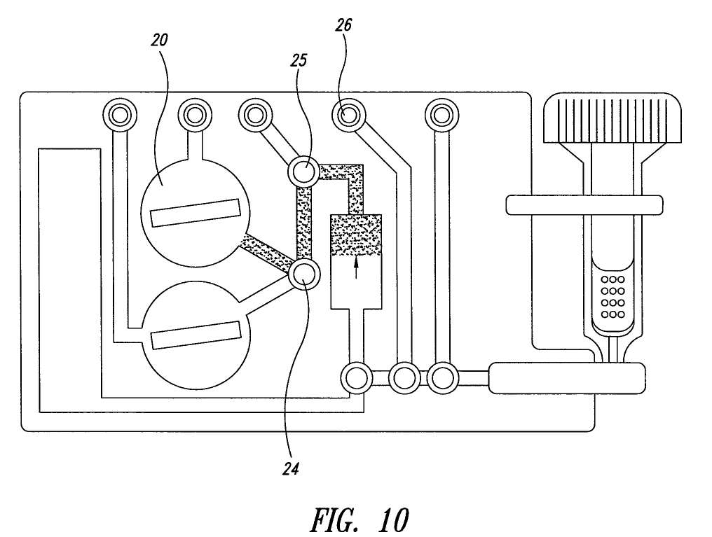

FIG. 10 shows the fourth step of the pre-programmed assay controlled by instrument 8, which is the elution of the sample RNA from the RNA isolation chamber to the test sample amplification and detection chamber 20 via valves 25 and 24. This elution step is performed with the aid of an elution buffer introduced via port 26 and via valves 13 and 14. This elution buffer is of a type which is able to release the RNA from the surface of the solid phase material in RNA isolation chamber 15.

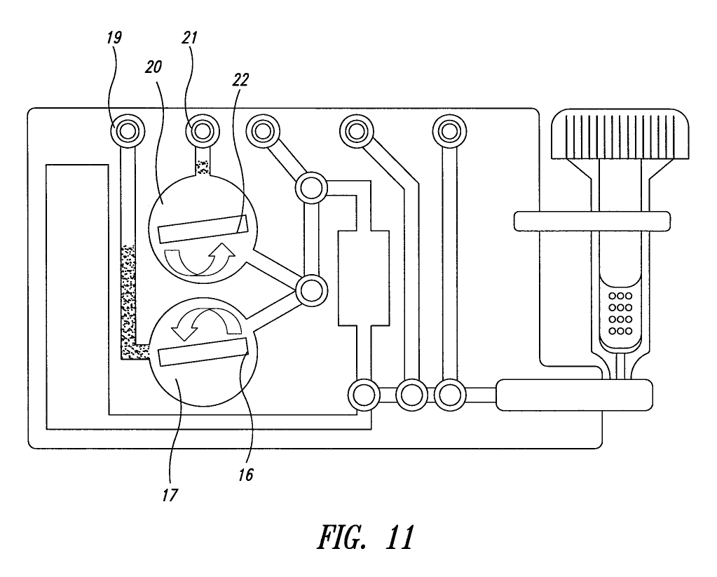

FIG. 11 shows the fifth step of the pre-programmed assay controlled by instrument 8, which is the dispensing of primer master mix for the target nucleic acid sequence into the test sample amplification and detection chamber 20 via port 21. Mixing of the primer master mix with the sample RNA is then performed by micro-magnetic stirrer bar 22. Further, a positive control, with control primers and template, for the target nucleic acid master mix is optionally dispensed into the positive control amplification and detection chamber 17 via port 19. Alternatively, a negative control may be run. Continued mixing of the positive control is then performed by micro-magnetic stirrer bar 16. Not shown in FIG. 11 is also an optional third negative control amplification and detection chamber which would be suitable for an FDA CLIA waived diagnostic device. In the negative control chamber de-ionised water would be introduced and mixed with the sample RNA, and no detection result would be expected after amplification. The quality control steps allowed by the positive and negative amplification and detection chambers are an essential step in gaining FDA CLIA waiver status, for embodiments where this might be required.

The make up of the reagents used in the master mix and positive control for the target nucleic acid sequence using the LAMP method is defined by the Eiken Chemical Co. Ltd of Japan. Such master mixes include primer mixes for a variety of infectious diseases, including H5 avian influenza for example.

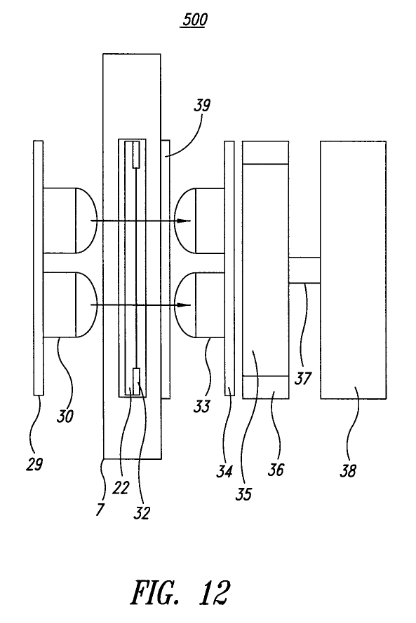

FIG. 12 shows how the amplification and detection is performed inside the test sample amplification and detection chamber 20. It should firstly be noted that the detection chamber 20 is transparent as the microfluidic device 7 is fabricated from an optically-transparent material. Instrument 8 includes a number of light emitting diodes (LEDs) 30 mounted onto a printed circuit board (PCB) 29. The LEDs 30 are adjacent to one side of the chamber 20, and shine collimated light through the chamber 20 in a direction which is orthogonal to the planar surface of the microfluidic device 7. The LEDs 30 are provided with a particular wavelength to suit subsequent turbidimetric detection.

The micro magnetic stirrer bar element 22 which is captured within chamber 20 is also constructed from an optically-transparent material. The outer edges of the stirrer bar element are printed with an iron-oxide material 32. This in turn allows a remote magnetic stirrer head 35 (to which is fitted outer magnets 36) to turn the stirrer bar element 22 inside the chamber thereby mixing the fluid contents contained within the chamber without disrupting the light path through the chamber provided by LEDs 30. Magnetic stirrer head 35 is driven by motor 38 via shaft 37, and this motor/stirrer head assembly is part of instrument 8.

On the reverse side of the test chamber a transparent Indium Tin Oxide (ITO) heating element 39 is printed onto the microfluidic device 7. This ITO heating element 39 makes an electrical contact with the instrument 8 in order to provide isothermal incubation to 62.5° C. as recommended for isothermal amplification for the LAMP method. Because the ITO heating element 39 is transparent, it does not disrupt the light path provided by LEDs 30.

Adjacent to the ITO heating element 39 is an array of photodiodes 33 mounted on a PCB 34 and which are part of instrument 8. The photodiodes 33 receive light emitted by the LEDs 30, and are able to detect the proportion of light that has been transmitted through chamber 30.

As the LAMP reaction proceeds, in the event of a positive test the amount of turbidity in the test sample amplification and detection chamber increases over time. After a known period of time, the turbidity level inside chamber 20 will increase to a level where photodiodes 33 are receiving a significantly lower proportion of light from LEDs 30 than they were at the start of the test. Conversely, in the event of a negative test, there will be no turbidity in the test chamber 20, and photodiodes 33 will receive the same proportion of light from LEDs 30 as at the start of the test. Thus, using a simple low cost turbidimetric detection approach, the system is able to diagnose and quantify the presence of the target nucleic acid sequence. Subsequent computer processing by instrument 8 is able to translate and display the results of the turbidimetric detection into clinically useful information which may be easily recorded or interpreted by a non-specialist operator.

The same process described above is also used in the positive control amplification and detection chamber 17 to verify that the assay has run correctly. Such a positive control step is a mandatory part of quality control in most molecular biology assays.

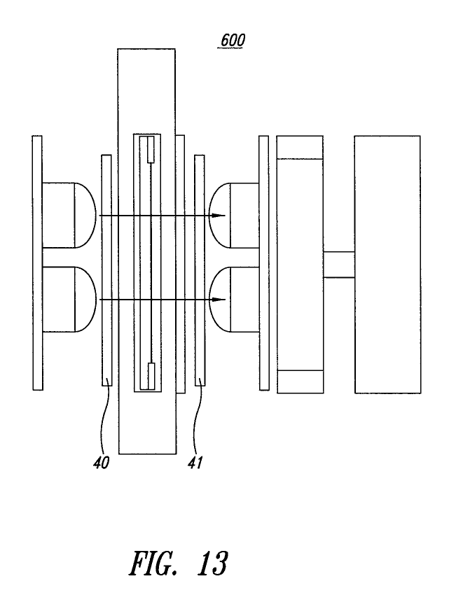

FIG. 13 shows an alternative detection embodiment inside the test sample amplification and detection chamber 20. In this case the Light-Emitting Diodes (LEDs) 30 are chosen to have an emission wavelength which corresponds to the absorbance wavelength of a fluorophore included in the master mix. These LEDs may shine through a thin film interference filter 40 (TFIF) which has a narrow bandpass and which allows light of only a short wavelength band to be transmitted through chamber 20.

The stirring and heating approach using this detection method is the same as was described for FIG. 12.

Light at the particular wavelength for the fluorophore of interest then causes the fluorophore to emit light at a different wavelength (the excitation wavelength) in the event that the target nucleic acid sequence is present and is undergoing amplification. This phenomenon where light is received by a fluorophore at one particular wavelength, and which causes the fluorophore to emit light at a second particular wavelength is known as a “Stoke's Shift”. The excitation light output may then also be passed through a second bandpass filter 41 prior to being received by photodiodes 33.

As the LAMP reaction proceeds, in the event of any light being received by photodiodes 33, a positive test result will be returned. Conversely, in the event of no light being received by photodiodes 33, a negative test result will be confirmed. The fluorometric detection approach may provide improved sensitivity over the turbidimetric detection method.

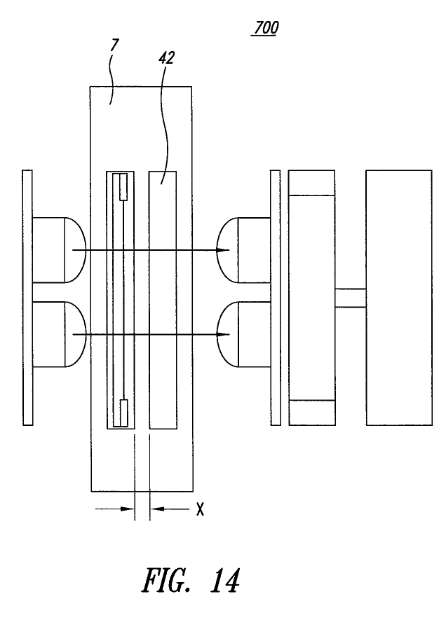

FIG. 14 shows an alternative heating approach for the polymerase reaction in which a secondary chamber 42 is provided within the microfluidic device 7. This chamber 42 is filled with either water or paraffin oil, which is heated in a separate zone to 62.5° C. by instrument 8 via a conventional heating element and recirculated within chamber 42. This heating approach may provide faster heating and more accurate temperature control than ITO heating element 39. Heating chamber 42, and the heating fluid (water or paraffin oil) are transparent so as not to block the light transmitted through the chamber. Alternatively they may be positioned so as not to obstruct a light path from LEDs 30 to photodiodes 33. Heating chamber 42 is also positioned at the minimum distance X from the chamber 20 in order to maximize heat transfer efficiency.

FIG. 15 shows a system block diagram of all the major elements of the diagnostic system, including the instrument 8, the reagent pack, and the microfluidic cartridge assembly 300 using the example of a reagent pack for the detection of H5 avian influenza. It should be noted from this diagram that instrument 8 may require on on-board cooling system (such as thermoelectric cooling elements) to keep the reagents in the reagent pack at a low storage temperature. Optionally, heat labile reagents such as polymerase and primer sets may be dehydrated and stored directly on the microfluidic cartridge 7, and then rehydrated in the elution buffer during the assay.

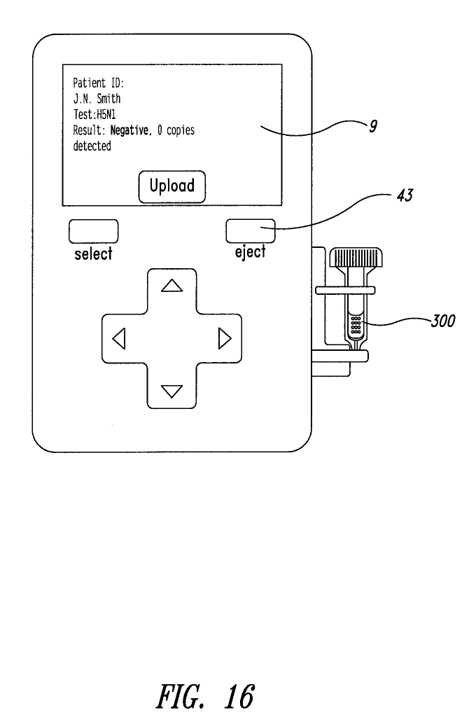

On completion of the test, the test result is displayed on the liquid crystalline display 9 of the instrument 8, thereby indicating whether the test result is positive or negative, and (in the event of a positive test) quantifying the amount of the virus or pathogen present. This is shown in FIG. 16. Such a result could optionally be communicated wirelessly to a central medical records database for the particular patient, provided that wireless communication means were built into instrument 8. The microfluidic cartridge assembly 300 (which contains the infectious sample) may then be ejected from instrument 8, and disposed into an approved bio-hazardous waste container.

The present invention thus provides for a single use disposable cartridge assembly 300 formed of the sample carrier 200 and microfluidic cartridge 7, which integrates the functions of sample preparation, nucleic acid extraction, amplification of target sequences, and detection of a target sequence. The disposable device works in conjunction with a control platform comprising portable instrument (reader) 8 and its reagent pack which provides the chemistry protocol to the disposable device in a pre-programmed manner thus avoiding the need for specialist involvement, and which stores and displays the results of the assay.

Thus, preferred embodiments of the present invention provide a portable or point of care bio-safe system for rapidly, reliably, and accurately detecting the target nucleic acid sequences of a range of infectious diseases, which is substantially as accurate as current PCR and RT-PCR based tests but which does not require expensive equipment, clinical laboratories, or skilled personnel to perform such tests. Furthermore, in embodiments where the system of the present invention is made portable, rapid in-field testing of particular infectious diseases may be performed.

Further, the United States Food and Drug Administration (FDA) in 1988 introduced guidelines for diagnostic systems that meet the requirements of the Clinical Laboratory Improvement Amendments (CLIA), which covers approximately 175,000 laboratory entities. CLIA defines a laboratory as any facility which performs laboratory testing on specimens derived from humans for the purpose of providing information for the diagnosis, prevention, treatment of disease, or impairment of, or assessment of health. Many clinicians' offices accordingly can now function as clinical laboratories by gaining CLIA waiver status. However to obtain CLIA waiver status, a diagnostic system must meet particular requirements of accuracy, sensitivity, quality control, and ease of use. Preferred embodiments of the present invention may thus provide for such waiver status.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

What is claimed is:

1. A biosafe system for assaying a target nucleic acid in a biosample, the system comprising:
a) A two-piece sample carrier comprising a swab for collecting a sample to be tested, said swab with capture end and extended neck topped by a threaded cap with locking means, and a body with compartment for accepting said swab, and further comprising a threaded upper lip and lower tubular nose with axial orifice, said orifice with inner seal;
b) A disposable microfluidic cartridge with external surfaces, with internal works, and with docking means for receiving said two-piece sample carrier, the microfluidic cartridge further comprising a bridging manifold with first fluidic channel in fluidic connection with a sample receiving receptacle, a means for sealingly accepting the tubular nose of said sample carrier in said sample receiving receptacle, a means for fluidically joining said first fluidic channel to said sample carrier, valve means for introducing and withdrawing lysis reagent to and from said compartment, a means for extracting a target nucleic acid from a sample lysate, a means for eluting a target nucleic acid, an amplification chamber and stirrer means for amplifying a nucleic acid in a sample eluate, a lightpath through said chamber for detecting an amplification product by optical detection means; and,
c) A control platform instrument with microprocessing means for sealedly engaging and controlling said internal works of said microfluidic cartridge, said means for sealingly engaging and controlling comprising at least one ported external hydraulic interface on said microfluidic cartridge, and detection means for reading and displaying an assay result; and further,
d) Wherein said means for sealingly accepting the tubular nose of said sample carrier in said sample receiving receptacle, said means for fluidically joining said first fluidic channel to said sample carrier, and said means for sealedly engaging and controlling said internal works are configured to isolate said nasal swab, internal works of said microfluidic cartridge, external surfaces, and instrument, from forward and reverse contamination.

2. A biosafe system of claim 1 wherein said means for sealingly accepting the tubular nose of said sample carrier in said sample receiving receptacle comprises a compression seal formed between said tubular nose with orifice and said sample receiving receptacle in said bridging manifold, said compression seal further comprising a snap-lock mechanism formed of a mating undercut locking ring in said sample receiving receptacle and an oversized barbed lip on said tubular nose with axial orifice, such that insertion of the barbed lip through said locking ring irreversibly secures said compression seal.

3. A biosafe system of claim 1 wherein said means for fluidically joining said first fluidic channel to said sample carrier comprises a snap-lock mechanism formed of a mating female locking ring in said sample receiving receptacle and an oversized barbed lip on said tubular nose, and further comprises a sharp mounted in said sample receiving receptacle of said bridging manifold and extending into said axial orifice of said tubular nose, whereby said sharp pierces said inner seal and forms a patent fluid path between said first fluidic channel of said sample receiving manifold and said sample body compartment containing said nasal swab as said sample carrier is pressed into said sample receiving receptacle of said bridging manifold, said press fit assembly further aided by docking means.

4. A biosafe system of claim 1, wherein said stirring means comprises a stirring motor with magnet on said control platform instrument and a stir bar with arms with ferromagnetic elements at the tips of said arms in said amplification chamber.

5. A biosafe system of claim 4, wherein said stir bar is transparent except at the tips of said arms.

6. A biosafe system of claim 1, wherein said optical detection means comprises an LED/photodiode pair straddling said optical window over said amplification chamber.

7. A biosafe system of claim 6, wherein said optical detection means further comprises an interference filter.

8. A biosafe system of claim 1, further comprising a resistive heating element contactingly disposed on said amplification chamber.

9. A biosafe system of claim 1, wherein said resistive heating element is a transparent ITO heating element.

10. A method for assaying a biosample for a target nucleic acid, the method comprising:
a) Collecting a sample with a swab and threadedly sealing said swab in a sample compartment in a sample carrier; said sample carrier further with tubular nose with central orifice, said orifice with inner seal; then,
b) Sealingly assembling said sample carrier into a sample receiving receptacle of a microfluidic cartridge, said sample receiving receptacle with piercing means, thereby piercing said inner seal and fluidically joining said sample compartment with a first fluidic channel of said microfluidic cartridge, thereby forming a microfluidics cartridge assembly; and thereafter,
c) Engaging said microfluidics cartridge assembly in a control platform instrument; and,
d) Sealedly introducing and withdrawing a lysis reagent to and from said sample compartment via said first fluidic channel, thereby forming a sample lysate; and aspirating said lysate into an isolation chamber on said microfluidics cartridge assembly; and therein,
e) Sealedly extracting a target nucleic acid from said sample lysate nucleic acid onto a solid phase matrix, thereby forming a solid phase retentate; and,
f) Sealedly eluting the target nucleic acid from said solid phase matrix, thereby forming an eluate; and further,
g) Sealedly amplifying said target nucleic acid with amplification reagents; before,
h) Sealedly detecting amplification products by optical detection means;
i) And further having controlled said steps of the assay by activating electrical and hydraulic control interfaces of said control instrument platform; before finally,
j) Disposing said microfluidics cartridge assembly.

11. The method of claim 10 wherein said amplification step comprises a LAMP protocol.

12. The method of claim 10, wherein said optical detection means comprises a step for hybridizing a probe with fluorophore.

13. The method of claim 10, wherein said optical detection means comprises a step for turbidometry.

14. The method of claim 10 wherein the nucleic acid target is a nucleic acid of a respiratory pathogen.

15. The method of claim 14 further comprising a step for reverse transcriptase mediated synthesis of cDNA from RNA of a respiratory pathogen.

16. The method of claim 10 wherein the nucleic acid target is a host genomic DNA.

17. The method of claim 10 further comprising a control reaction run side-by-side with the bioassay.

18. The method of claim 10 wherein said amplification reagents are provided on-cartridge as dehydrated reagents.

19. The biosafe system of claim 1, wherein the microfluidics cartridge assembly and control platform instrument combination is portable.

20. The steps, features, integers, compositions and/or compounds disclosed herein or indicated in the specification of this application individually or collectively, and any and all combinations of two or more of said steps or features.
